(19) United States
(12) Patent Application Publication
(10) Pub. No.: US 2026/0071206 A1
(43) Pub. Date: Mar. 12, 2026

(54) COMPOSITIONS, KITS, AND METHODS FOR ANALYSIS OF DNA SEQUENCE-SPECIFICITY IN V(D)J RECOMBINATION

(71) Applicant: The Board of Regents of the University of Oklahoma, Norman, OK (US)

(72) Inventors: Karla RODGERS, Edmond, OK (US); Walker HOOLEHAN, Oklahoma City, OK (US)

(21) Appl. No.: 19/038,963

(22) Filed: Jan. 28, 2025

Related U.S. Application Data
(62) Division of application No. 17/683,744, filed on Mar. 1, 2022, now abandoned.
(60) Provisional application No. 63/160,136, filed on Mar. 12, 2021.

(51) Int. Cl. C12N 15/10; C12N 5/071; C12N 9/22; C12N 15/85; C12P 19/34; C12Q 1/6806; C12Q 1/6869

(52) U.S. Cl. CPC C12N 15/1065; C12N 5/0687; C12N 9/22; C12N 15/1093; C12N 15/85; C12P 19/34; C12Q 1/6806; C12Q 1/6869; C12N 2800/107

(57) ABSTRACT

Compositions, kits, systems, and methods are disclosed for use in analysis of DNA sequence-specificity in V(D)J recombination or other types of recombination. In particular (but not by way of limitation), high throughput methods of analyzing DNA sequence-specificity in a V(D)J recombination assay are disclosed.

### CROSS REFERENCE TO RELATED APPLICATIONS/INCORPORATION BY REFERENCE STATEMENT

The present application is a divisional of U.S. patent application Ser. No. 17/683,744, filed Mar. 1, 2022; which claims the benefit under 35 USC § 119(e) of U.S. Provisional Patent Application Ser. No. 63/160,136, filed Mar. 12, 2021, the disclosure of which is herein incorporated by reference in its entirety.

### STATEMENT REGARDING FEDERALLY SPONSORED RESEARCH OR DEVELOPMENT

This invention was made with government support under Award Number AI156351 awarded by the National Institutes of Health. The government has certain rights in the invention.

### REFERENCE TO SEQUENCE LISTING SUBMITTED ELECTRONICALLY

The instant application contains, as a separate part of the present disclosure, a Sequence Listing which has been submitted via EFS-Web in computer readable form as an XML file. The Sequence Listing, created Jan. 27, 2025, is named “5864172 Sequence Listing.xml” and is 21,271 bytes in size. The entire contents of the Sequence Listing are hereby incorporated herein by reference.

### BACKGROUND

The adaptive immune system is a critical line of defense against the onslaught of pathogenic organisms and viruses that our bodies are exposed to on a daily basis. B and T cell populations expressing diverse antigen receptors, which have the collective ability to recognize a vast array of foreign antigens, mediate the immune response. The diverse receptors are formed during lymphocyte development through shuffling of individual gene segments in the antigen receptor loci in a process known as V(D)J recombination. Antigen receptor gene segments are termed V (variable), D (diversity), and J (joining) and are marked for potential recombination events by a flanking recombination signal sequence (RSS). The RSS (SEQ ID NO:1) consists of conserved heptamer and nonamer sequences separated by 12 or 23 base pairs (referred to as the 12-RSS and 23-RSS, respectively; see FIG. 1, Panel A). Recombination preferentially occurs between two segments flanked by RSSs with differing spacer lengths, a restriction referred to as the 12/23 rule. The 12/23 rule maintains the correct ordered assembly of the gene segments to yield joined V-D-J gene segments, or V-J in antigen receptor loci lacking D segments. Thus, based on the arrangement of the gene segments and the type of flanking RSS (12-RSS or 23-RSS), the 12/23 rule serves to prevent joining of the same class of gene segment (i.e. V-V) or incorrect combinations (i.e. V-J in gene loci containing D segments).

The recombination activating gene proteins, RAG1 and RAG2, initiate V(D)J recombination by generating DNA double strand breaks (DSBs) at the border of the gene segment and flanking RSS through a two-step nicking and hairpin formation mechanism. RAG-mediated DNA DSBs occur in the context of a paired complex (PC), with the RAG proteins simultaneously bound to a 12-RSS and a 23-RSS with the intervening DNA looped out (FIG. 1, Panel B). Both RAG proteins are required for DNA cleavage activity, with RAG1 containing the active site residues, as well as the RSS specific binding sites. The role for RAG2 is less clear, but may function to activate RAG1 for sequence-specific binding and cleavage, and also provide additional DNA binding capability. Following RAG-mediated DNA cleavage, the appropriate DNA ends are joined by the action of ubiquitous DNA repair factors that function in nonhomologous DNA end-joining (NHEJ). Erroneous RAG-mediated DNA cleavage, either at RSS-like sites or non-B form structures, as well as mistakes in DNA repair, are known to result in chromosomal translocations with increased risk for development of certain leukemias and lymphomas.

Various high throughput methods have been developed to identify DNA sequences recognized or cleaved by specific proteins. In addition, NGS methods have been developed to identify rearranged genomic products or DNA cleavage sites that form during V(D)J recombination. Each of these methods is discussed herein below.

Bind-n-Seq (BNS) is a method developed by Zykovich et al. (Nucleic Acids Res (2009) 37, e151) to identify specific DNA sequences that are recognized and bound by a protein-of-interest (POI). In this method, synthetic duplexed oligonucleotides containing a window of degenerate base pairs is incubated in vitro with the purified POI, and the POI bound to preferred DNA sequences is separated from unbound oligonucleotide duplexes. The bound DNA is released from the POI and subjected to next generation sequencing. Analysis of the sequenced DNA provides preferred sequence motifs that the POI binds. However, this method has several disadvantages, including that the method must be performed in vitro with purified components. Further, the method determines sequence-specific DNA binding activity and is designed for use with transcription factors. Therefore, the application of the Bind-n-Seq method is not optimal for analyzing specific sequences of DNA that are recognized and cleaved by enzymes.

NucleaSeq (nuclease digestion and sequencing) was recently developed by Jones et al. (bioRxiv 696393 (2019) doi: 10.1101/696393) to measure the cleavage kinetics of CRISPR-Cas nucleases.

V(D)J recombination leads to the immune repertoire. The immune repertoire is typically analyzed at the RNA level by RNA seq. V(D)J recombination events at the DNA level (instead of relying on RNA transcripts) is analyzed by V(D)J-seq and high-throughput genome-wide translocation sequencing (HTGTS) methods (see, for example, (Chovanec et al. (Nat Protoc (2018) 13, 1232-1252) and Lin et al. (Proc Natl Acad Sci USA (2016) 113, 7846-7851)). These methods show what gene segments are combined in V(D)J recombination. Comparison to the germline sequence is used to determine the sequence of the RSS that had adjoined to the gene segments prior to the recombination event. However, these methods do not provide an unbiased analysis of the DNA sequence specificity of the V(D)J recombinase, since only endogenous RSSs are analyzed. Endogenous RSSs differ in their chromatin environment from one another, complicating the analysis of DNA sequence specificity by the RAG proteins.

END-seq is a method developed by Canela et al. (Mol Cell (2016) 63, 898-911) to identify DNA sequences at DNA double strand breaks. This method has been applied to analysis of RAG-mediated breaks during V(D)J recombination. However, as described herein above with reference to the methods of Chovanec et al. and Lin et al., this method can only be applied to endogenous RSSs, which are not in a uniform sequence background.

Therefore, there is a need in the art for new and improved compositions and methods that overcome the disadvantages and defects of the prior art. It is to such compositions and methods that the present disclosure is directed.

### BRIEF DESCRIPTION OF THE DRAWINGS

FIG. 1 is a schematic showing the structure of RAG-RSS complexes formed during V(D)J recombination. Panel (A)—The accepted canonical (i.e., consensus) sequences for the heptamer and nonamer portions of the RSS (SEQ ID NO:1). Only the DNA strand initially nicked by the RAG proteins is shown. Nicking occurs 5′ to the heptamer at the position indicated. Panel (B)—RAG complexes formed in the V(D)J recombination reaction. R1 and R2 refer to RAG1 and RAG2, respectively. Circles labeled H (in the PC, CSC, and SEC) refer to HMGB1 or HMGB2. (Reprinted from Rodgers, Trends Biochem Sci. (2017) 42(1): 72-84).

FIG. 2 is a schematic showing a V(D)J recombination reaction between D and J gene segments in the IgH locus. The top reaction, catalyzed by RAG1 and RAG2, results in DNA double strand breaks between each RSS and its adjoining gene segment. The bottom reaction represents DNA repair by non-homologous end-joining factors, and leads to imprecise joining of the DH and JH gene segments (coding joint) and a heptamer-to-heptamer junction between the 12-RSS and 23-RSS (signal joint). In the orientation shown here, the signal joint is deleted from the chromosome.

FIG. 3 is a schematic showing the plasmid recombination assay. The plasmid contains a 12-RSS and a 23-RSS in a colinear orientation, as shown in the left reaction. In the middle reaction, RAG1 and RAG2 create DNA double strand breaks between the heptamer in each RSS and its adjoining DNA. In the right reaction, NHEJ factors invert the resulting DNA fragment and ligate the ends to form the signal joint and the coding joint. The arrows show the orientation of the PCR primers used to amplify the recombined products. The arrow head is the 3′OH end of each PCR primer. With successful recombination, the PCR primers will be in the proper orientation to generate a PCR product of known size.

FIG. 4 shows an embodiment of a construct of the present disclosure. Panel (A)—The top strand of the 12-RSS (SEQ ID NO:2), indicating the position of the 6 consecutive degenerate base pairs (Ns). Panel (B)—Capillary DNA sequencing of the plasmid input library. The degenerate bases are small letter Ns. The sequence shown has been assigned SEQ ID NO:3 and is the reverse complement of the sequence in panel A.

FIG. 5 shows SARP-seq results showing the fractional abundance of unique RSSs (left panel). The input library contains equal proportions of the 4 bases at each of the 6 degenerate positions. The relative abundance of unique RSSs after recombination (output library) is plotted above. The dot at the far right (at ˜0.013) is the top read at 18, 804. Sequences with high read counts (over 10,000 in the pilot experiment) are shown in the right panel. In the pilot experiment, there were 1261 unique sequences with reads >100.

FIG. 6 is a schematic showing an inversion reaction. In Panel A, the plasmid substrate for SARP-seq contains a UMI (as bold green line) adjacent to the 12-RSS (red triangle). After V(D)J recombination and isolation of the plasmid, the recombined plasmid is the template for PCR. The final PCR product (output library) is shown schematically and expanded in Panel B. The sequence shown in Panel B has been assigned SEQ ID NO:4.

FIG. 7 is a diagram showing precise versus imprecise signal joints. SARP-seq identifies variant RSSs that are found at higher frequency in imprecise (SEQ ID NOS:6 and 7) versus precise (SEQ ID NO:5) signal joints.

FIG. 8 is a schematic showing Hybrid joint formation of the SARP-seq input plasmid leads to deletion of a fragment that includes the 23-RSS and the intervening DNA to the 12-RSS. The 12-RSS is joined to the DNA that had previously bordered the 23-RSS, thus forming the hybrid joint. The smaller PCR product containing the hybrid joint is readily separated using gel electrophoresis from the PCR product of unrearranged input plasmid. NGS analysis shows if certain RSS sequences preferentially occur in hybrid joints.

FIG. 9 is a schematic showing SARP-seq with degenerate DNA split between the 12-RSS heptamer and nonamer. Sequence shown therein has been assigned SEQ ID NO:8.

FIG. 10 is a schematic showing SARP-seq with degenerate 12-RSSs and 23-RSSs. Right panel: top sequence has been assigned SEQ ID NO:9; middle sequence has been assigned SEQ ID NO:10; and bottom sequence has been assigned SEQ ID NO:11.

FIG. 11 is a schematic showing coding joint analysis by SARP-seq. Here, primers are designed to flank the coding sequences (the DNA flanking the RSSs). Amplification of the coding joint will occur only in the recombined plasmids. The final PCR product containing the coding joint will be analyzed by NGS. Due to the inaccuracy of NHEJ, NGS analysis of the output library will allow sorting of products with variable numbers of bases added or deleted at the coding joint.

### DETAILED DESCRIPTION

The present disclosure is directed to compositions, kits, systems, and methods for analyzing DNA sequence-specificity in various types of recombination reactions. In particular (but non-limiting) embodiments, the present disclosure describes a new method, referred to as Selective Amplification of Recombination Products with sequencing (SARP-seq). The method was designed to investigate the DNA sequence-specificity that is fundamental to V(D)J recombination, a process that assembles functional antigen receptor genes during B and T cell development. The V(D)J recombinase components, RAG1 and RAG2, cleave adjacent to recombination signal sequences (RSSs) that flank coding gene segments in the antigen receptor loci. After RAG-mediated DNA cleavage, DNA repair factors join the gene segments together to form the coding sequence for the antigen receptor.

The embodiments of the present disclosure are not limited to the details of construction and the arrangement of the components set forth in the following description and are capable of other embodiments or of being practiced or carried out in various ways. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting.

Unless otherwise defined herein, scientific and technical terms used in the present disclosure shall have the meanings that are commonly understood by those of ordinary skill in the art. Further, unless otherwise required by context, singular terms shall include pluralities and plural terms shall include the singular. The foregoing techniques and procedures are generally performed according to conventional methods well known in the art and as described in various general and more specific references that are cited and discussed throughout the present specification. The nomenclatures utilized in connection with, and the laboratory procedures and techniques of, analytical chemistry, synthetic organic chemistry, cell and tissue culture, molecular biology, and protein and oligo- or polynucleotide chemistry, and medicinal and pharmaceutical chemistry described herein are those well-known and commonly used in the art. Standard techniques are used for recombinant DNA, oligonucleotide synthesis, and tissue culture and transformation (e.g., electroporation, lipofection). Enzymatic reactions and purification techniques are performed according to manufacturer's specifications or as commonly accomplished in the art or as described herein. The foregoing techniques and procedures are generally performed according to conventional methods well known in the art and as described in various general and more specific references that are cited and discussed throughout the present specification.

All patents, published patent applications, and non-patent publications mentioned in the specification are indicative of the level of skill of those skilled in the art to which the present disclosure pertains. All patents, published patent applications, and non-patent publications referenced in any portion of this application are herein expressly incorporated by reference in their entirety to the same extent as if each individual patent or publication was specifically and individually indicated to be incorporated by reference.

While the compositions and methods of the present disclosure have been described in terms of particular embodiments, it will be apparent to those of skill in the art that variations, substitutions, and modifications may be applied to the compositions and/or methods and in the steps or in the sequence of steps of the methods described herein without departing from the spirit and scope of the inventive concepts disclosed herein, for example as defined in, but not limited to, the appended claims, which are presented herein as exemplary only.

As utilized in accordance with the present disclosure, the following terms, unless otherwise indicated, shall be understood to have the following meanings:

The use of the term “a” or “an” when used in conjunction with the term “comprising” in the claims and/or the specification may mean “one,” but it is also consistent with the meaning of “one or more,” “at least one,” and “one or more than one.” As such, the terms “a,” “an,” and “the” include plural referents unless the context clearly indicates otherwise. Thus, for example, reference to “a compound” may refer to one or more compounds, two or more compounds, three or more compounds, four or more compounds, or greater numbers of compounds. The term “plurality” refers to “two or more.”

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, includes ranges of 1-20, 10-50, 50-100, 100-500, and 500-1,000, for example. Reference to an integer with more (greater) or less than includes any number greater or less than the reference number, respectively. Thus, for example, reference to less than 100 includes 99, 98, 97, etc. all the way down to the number one (1); and less than 10 includes 9, 8, 7, etc. all the way down to the number one (1).

The use of the term “at least one” will be understood to include one as well as any quantity more than one, including but not limited to, 2, 3, 4, 5, 10, 15, 20, 30, 40, 50, 100, etc. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y, and Z” will be understood to include X alone, Y alone, and Z alone, as well as any combination of X, Y, and Z. The use of ordinal number terminology (i.e., “first,” “second,” “third,” “fourth,” etc.) is solely for the purpose of differentiating between two or more items and is not meant to imply any sequence or order or importance to one item over another or any order of addition, for example.

The use of the term “or” in the claims is used to mean an inclusive “and/or” unless explicitly indicated to refer to alternatives only or unless the alternatives are mutually exclusive. For example, a condition “A or B” is satisfied by any of the following: A is true (or present) and B is false (or not present), A is false (or not present) and B is true (or present), and both A and B are true (or present).

As used herein, any reference to “one embodiment,” “an embodiment,” “some embodiments,” “one example,” “for example,” or “an example” means that a particular element, feature, structure, or characteristic described in connection with the embodiment is included in at least one embodiment. The appearance of the phrase “in some embodiments” or “one example” in various places in the specification is not necessarily all referring to the same embodiment, for example. Further, all references to one or more embodiments or examples are to be construed as non-limiting to the claims.

Throughout this application, the terms “about” or “approximately” are used to indicate that a value includes the inherent variation of error for the composition, the method used to administer the composition, or the variation that exists among the study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The terms “about” or “approximately,” where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art.

As used in this specification and claim(s), the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

As used herein, the term “substantially” means that the subsequently described event or circumstance completely occurs or that the subsequently described event or circumstance occurs to a great extent or degree. For example, when associated with a particular event or circumstance, the term “substantially” means that the subsequently described event or circumstance occurs at least 80% of the time, or at least 85% of the time, or at least 90% of the time, or at least 95% of the time. For example, the term “substantially adjacent” may mean that two items are 100% adjacent to one another, or that the two items are within close proximity to one another but not 100% adjacent to one another, or that a portion of one of the two items is not 100% adjacent to the other item but is within close proximity to the other item.

The term “polynucleotide” or “oligonucleotide” as used herein will be understood to refer to a polymer of two or more nucleotides. Nucleotides, as used herein, will be understood to include deoxyribose nucleotides and/or ribose nucleotides, as well as artificial variants thereof. The term polynucleotide also includes single-stranded and double-stranded molecules.

The terms “analog” or “variant” as used herein will be understood to refer to a variation of the normal or standard form or the wild-type form of molecules. For polypeptides or polynucleotides, an analog may be a variant (polymorphism), a mutant, and/or a naturally or artificially chemically modified version of the wild-type polynucleotide (including combinations of the above). Such analogs may have higher, full, intermediate, or lower activity than the normal form of the molecule, or no activity at all. Alternatively, and/or in addition thereto, for a chemical, an analog may be any structure that has the desired functionalities (including alterations or substitutions in the core moiety), even if comprised of different atoms or isomeric arrangements.

As used herein, the phrases “associated with” and “coupled to” include both direct association/binding of two moieties to one another as well as indirect association/binding of two moieties to one another. Non-limiting examples of associations/couplings include covalent binding of one moiety to another moiety either by a direct bond or through a spacer group, non-covalent binding of one moiety to another moiety either directly or by means of specific binding pair members bound to the moieties, incorporation of one moiety into another moiety such as by dissolving one moiety in another moiety or by synthesis, and coating one moiety on another moiety, for example.

As used herein, “pure” or “substantially pure” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure.

The term “active agent” as used herein is intended to refer to a substance which possesses a biological activity relevant to the present disclosure, and particularly refers to therapeutic and diagnostic substances which may be used in methods described in the present disclosure. By “biologically active” is meant the ability to modify the physiological system of a cell, tissue, or organism without reference to how the active agent has its physiological effects.

As noted above, certain non-limiting embodiments directed to compositions, kits, systems, and methods for analyzing DNA sequence-specificity in various types of recombination reactions are disclosed herein. In particular (but non-limiting) embodiments, the present disclosure describes a new method, referred to as Selective Amplification of Recombination Products with sequencing (SARP-seq). The method was designed to investigate the DNA sequence-specificity that is fundamental to V(D)J recombination, a process that assembles functional antigen receptor genes during B and T cell development. The V(D)J recombinase components, RAG1 and RAG2, cleave adjacent to recombination signal sequences (RSSs) that flank coding gene segments in the antigen receptor loci. After RAG-mediated DNA cleavage, DNA repair factors join the gene segments together to form the coding sequence for the antigen receptor. RSSs are only partially conserved, and many RSS-like sequences are present elsewhere in the genome. Principles that dictate DNA sequence specificity of the V(D)J recombinase for bona fide RSSs are not known, since the majority of studies have focused on interaction of the RAG proteins with only a few different RSSs using low-throughput approaches. Compositions, kits, and methods have been developed to investigate RAG-RSS interactions using an unbiased, high-throughput approach. First, a plasmid recombination assay that uses a plasmid substrate containing two RSSs was modified. Successful recombination leads to inversion of a segment of DNA in the plasmid. In the modified assay, a window of fully degenerate consecutive base pairs was inserted within one of the RSSs of the plasmid substrate, thus introducing thousands of potential sequences that can be tested simultaneously in the recombination assay. The plasmid is then transfected into cells co-expressing the RAG proteins. Following a specified time period to allow recombination to occur, plasmid is recovered. The recombined plasmid is selectively amplified by PCR using primers that will only amplify the inverted recombination product; hence, selective amplification of recombination products (SARP). The resulting PCR product is subsequently analyzed by next generation sequencing. Example 1 includes a proof-of-principle experiment that demonstrates the ability to delineate a hierarchy of sequence motifs utilized by the V(D)J recombinase, as well as interrelationships between DNA base pairs that influences the relative level of recombination. This method is flexible in design in that recombination side-products can also be analyzed using the same input substrate. In addition, modifications of the plasmid substrate may be used for analysis of DNA sequence specificity with other enzymes that act on DNA.

This novel method for investigating RSS selectivity in V(D)J recombination provides various advantages, including that RSS quality in the complete V(D)J recombination reaction can be evaluated for thousands to millions of possible DNA sequences. No other methods are available that investigates the DNA selectivity of the V(D)J recombination reaction in an unbiased, high throughput manner. Another advantage of this method is the generation of the plasmid substrate containing degenerate sequences. This permits the simultaneous analysis of thousands, and potentially millions, of potential recombination substrates through next generation sequencing methods. Yet another advantage of this method is that recombined products can be selected from the vast majority of unrecombined plasmid by selectively amplifying the inverted portion of recombined plasmids. Further, sequence data can be readily sorted based on the presence of the signal joint, the molecular signature of V(D)J recombination.

The plasmid input library as described herein can be utilized to test the effect of changes in the sequence of one or both RSS's on RAG-RSS interactions and recombination. The plasmid input library can also be utilized to test the effect of mutations of proteins that function in V(D)J recombination on DNA selectivity in the reaction. Non-limiting examples include using the plasmid library to test DNA selectivity of RAG1 and RAG2 mutants that are suspected of causing immune system disorders.

The present disclosure allows for the production of plasmid input libraries that are specially designed based on an investigator's specifications. These products could be used (for example, but not by way of limitation) by investigators studying nucleic acid enzymes that rearrange DNA through inversion reactions. This includes V(D)J recombination and other DNA transposase systems. The present disclosure allows for the performance of one or more of the following: 1) the plasmid recombination assay, 2) preparation of the PCR product, 3) next generation sequencing, and 4) data analysis.

The compositions and methods of the present disclosure can be utilized (for example, but not by way of limitation) in the fields of immunology, immune disorders, DNA repair, genomic analysis of lymphomas and leukemias, and the like. Industries include those that provide DNA substrates for various purposes.

Certain non-limiting embodiments of the present disclosure are directed to a plasmid library that comprises a plurality of plasmid constructs. Each of the plasmid constructs comprises a plasmid vector having a 12-recombination signal sequence (12-RSS) and a 23-recombination signal sequence (23-RSS) inserted therein in a colinear orientation and a segment of at least 100 base pairs inserted in between the 12-RSS and the 23-RSS. The 12-RSS comprises a heptamer, a 12 base pair spacer, and a nonamer, and the 23-RSS comprises a heptamer, a 23 base pair spacer, and a nonamer. At least one of the 12-RSS and the 23-RSS has a degenerate base pair sequence of one to 25 (or more) consecutive base pairs present therein. In addition, the plasmid library comprises about 4ⁿ plasmids, wherein n is the number of base pairs present in the degenerate base pair sequence.

That, is the 12-RSS and/or the 23-RSS may have a degenerate base pair sequence of about 1, about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, or more consecutive base pairs therein. When the degenerate base pair sequence contains 1 base pair, the plasmid library comprises about 41 or 4 plasmids (each with one of A, G, C, or T at the single base pair position). When the degenerate base pair sequence contains 2 base pairs, the plasmid library comprises about 42 or 16 plasmids (for each possible combination of A, G, C, or T at the two locations). Similarly, 3 base pairs in the degenerate sequence provides about 4³ (64) plasmids, 4 base pairs in the degenerate sequence provides about 4⁴ (256) plasmids, 5 base pairs in the degenerate sequence provides about 4⁵ (1024) plasmids, 6 base pairs in the degenerate sequence provides about 4⁶ (4,096) plasmids, etc.

Any plasmids known in the art or otherwise contemplated herein that are capable of functioning as described herein may be utilized in accordance with the present disclosure. One non-limiting example of a plasmid vector is a pMX-based vector, such as, but not limited to, pMX-INV (also known as pMX-RSS-EGFP/IRES-hCD4; see, for example, Bredemeyer et al. (Nature (2006), Vol. 442, 466-470); and Liang et al. (Immunity (2002) Vol. 17, 639-651)).

The native/canonical sequence of the heptamers of the 12-RSS and 23-RSS may be CACAGTG, while the native/canonical sequence of the nonamers of the 12-RSS and 23-RSS may be ACAAAAACC. In general, the CAC sequence of the heptamer is highly conserved and is not included in the degenerate base pair sequence. However, any other portion of the heptamer, the spacer, and/or the nonamer may be included in the degenerate base pair sequence.

In particular (but non-limiting) embodiments, the degenerate base pair sequence comprises 2 to 25 consecutive base pairs that span at least a portion of the heptamer and at least a portion of the spacer of the 12-RSS or 23-RSS. In a particular (but non-limiting) embodiment, the degenerate base pair sequence comprises 2 to 10 consecutive base pairs that span at least a portion of the heptamer and at least a portion of the spacer of the 12-RSS or 23-RSS.

In other particular (but non-limiting) embodiments, the degenerate base pair sequence comprises 2 to 25 base pair changes that span at least a portion of the spacer and at least a portion of the nonamer of the 12-RSS or 23-RSS. In a particular (but non-limiting) embodiment, the degenerate base pair sequence comprises 2 to 10 base pair changes that span at least a portion of the spacer and at least a portion of the nonamer of the 12-RSS or 23-RSS.

In one particular (but non-limiting) embodiment, the degenerate base pair sequence comprises the AGTG positions of the heptamer and at least a portion of the spacer of the 12-RSS or 23-RSS.

In certain particular (but non-limiting) embodiments, degenerate base pair sequences may be present in both RSS's. That is, each of the 12-RSS and the 23-RSS may have a degenerate base pair sequence of one to 25 consecutive base pairs present therein (such as, but not limited to, 1 to 10 consecutive base pairs present therein). In this embodiment, the plasmid library comprises about 4ⁿ plasmids, wherein n is the total number of base pairs present in the combination of the two degenerate base pair sequences.

Certain non-limiting embodiments of the present disclosure are directed to a method of producing any of the plasmid libraries disclosed or otherwise contemplated herein. The method comprises the steps of: (1) producing a plurality of synthetic oligonucleotides that comprise the RSS having the degenerate base pair sequence of one to 25 consecutive base pairs present therein (such as, but not limited to, 1 to 10 consecutive base pairs or 2 to 10 consecutive base pairs therein), wherein the plurality of synthetic oligonucleotides comprises about 4ⁿ oligonucleotides, wherein n is the number of base pairs present in the degenerate base pair sequence; (2) converting the plurality of synthetic oligonucleotides to double-stranded DNA; (3) linearizing a plasmid, wherein the plasmid comprises canonical 12-RSS and canonical 23-RSS present therein in a colinear orientation and the segment of at least 100 base pairs disposed therebetween; (4) removing the canonical RSS that corresponds to the plurality of synthetic oligonucleotides; and (5) ligating the double-stranded DNA comprising the plurality of synthetic oligonucleotides to the plasmid to produce the plasmid library.

In certain embodiments of the method, the plasmid vector may be pMX-INV. The degenerate base pair sequence may contain, for example, one to ten consecutive base pairs therein. A native sequence of the heptamers of the canonical 12-RSS and 23-RSS may be CACAGTG, and a native sequence of the nonamers of the canonical 12-RSS and 23-RSS may be ACAAAAACC. In certain embodiments, the degenerate base pair sequence does not include the CAC sequence of the heptamer. The degenerate base pair sequence may comprise two to ten consecutive base pairs that span at least a portion of the heptamer and at least a portion of the spacer of the 12-RSS or 23-RSS. The degenerate base pair sequence may comprise two to ten base pair changes that span at least a portion of the spacer and at least a portion of the nonamer of the 12-RSS or 23-RSS. The degenerate base pair sequence may comprise the AGTG positions of the heptamer and at least a portion of the spacer of the 12-RSS or 23-RSS.

When two degenerate base pair sequences are utilized (one in each RSS), step (1) of the method will be further defined as: (la) producing a first plurality of synthetic oligonucleotides that comprise the 12-RSS having the degenerate base pair sequence of one to 25 consecutive base pairs present therein (such as, but not limited to, 1 to 10 consecutive base pairs or 2 to 10 consecutive base pairs therein), wherein the first plurality of synthetic oligonucleotides comprises about 4n oligonucleotides, wherein n is the number of base pairs present in the degenerate base pair sequence; and (lb) producing a second plurality of synthetic oligonucleotides that comprise the 23-RSS having the degenerate base pair sequence of one to 25 consecutive base pairs present therein (such as, but not limited to, 1 to 10 consecutive base pairs or 2 to 10 consecutive base pairs therein), wherein the second plurality of synthetic oligonucleotides comprises about 4n oligonucleotides, wherein n is the number of base pairs present in the degenerate base pair sequence. The first and second pluralities of synthetic oligonucleotides will both be converted to double-stranded DNA and ligated into the linearized plasmid to produce the plasmid library.

Certain non-limiting embodiments of the present disclosure are directed to a high throughput method of analyzing DNA sequence-specificity in a V(D)J recombination assay (or any other type of recombination assay). The method comprises the steps of: (1) transfecting mammalian cells with any of the plasmid libraries disclosed or otherwise contemplated herein, wherein the mammalian cells are capable of expressing recombination activating gene proteins 1 and 2 (RAG1 and RAG2); (2) culturing the transfected cells under conditions that allow for expression of RAG1 and RAG2 and production of recombination products in which the portion of the plasmids between the 12-RSS and 23-RSS is inverted to form a 12-RSS:23-RSS signal joint and a coding joint; (3) harvesting the transfected mammalian cells; (4) recovering plasmid DNA from the harvested cells; and (5) selectively amplifying the recombination products using primers that amplify the 12-RSS:23-RSS signal joint formed during recombination, wherein the selectively amplified recombination products constitute an output library for the recombination assay.

In particular (but non-limiting) embodiments, the method additionally comprises the steps of: (6) sequencing the output library from the recombination assay; and (7) analyzing the degenerate base pair sequences present in the selectively amplified signal joints of the output library.

Any mammalian cells known in the art or otherwise contemplated herein may be utilized in accordance with the present disclosure, as long as the mammalian cells are capable of functioning as described herein. One non-limiting example thereof is HEK293 cells.

The mammalian cells may endogenously express RAG1 and/or RAG2. Alternatively, and/or in addition thereto, the mammalian cells may be transfected with a single expression vector encoding both RAG1 and RAG2, and/or at least one expression vector encoding RAG1 and at least one expression vector encoding RAG2.

The methods of the present disclosure also allow for the analysis of DNA sequence-specificity of one or more RAG mutants in a V(D)J recombination assay; this method is particularly useful in the study of immunodeficiency diseases that are known to have one or more RAG mutations associated therewith. In this embodiment, the mammalian cells do not endogenously express at least one of the RAGs of interest, and the mammalian cells are transfected with an expression vector containing the mutated RAG of interest.

Certain non-limiting embodiments are directed to a single plasmid construct that comprises a plasmid vector; a 12-recombination signal sequence (12-RSS) inserted in the plasmid vector, wherein the 12-RSS comprises a heptamer, a 12 base pair spacer, and a nonamer; a 23-recombination signal sequence (23-RSS) inserted in the plasmid vector, wherein the 23-RSS comprises a heptamer, a 23 base pair spacer, and a nonamer; and a segment of at least 100 base pairs inserted in the plasmid vector in between the 12-RSS and the 23-RSS. The 12-RSS and 23-RSS are disposed in the plasmid vector in a colinear orientation, and at least a portion of at least one of the 12-RSS and the 23-RSS has a mutation when compared to a native 12-RSS or 23-RSS sequence, and the mutation comprises one to 25 consecutive base pair changes (such as, but not limited to, 1 to 10 consecutive base pair changes or 2 to 10 consecutive base pair changes therein) when compared to the native 12-RSS and 23-RSS sequence.

The plasmid construct may be produced in the same manner and include the same components as the plasmid library, except that the RSS mutation is a single example of a degenerate base pair sequence as described herein above with reference to the plasmid library.

In a non-limiting embodiment, the plasmid vector may be pMX-INV. In non-limiting embodiments, the mutation of the plasmid construct may comprise one to ten consecutive base pair changes. In non-limiting embodiments, the native sequence of the heptamers of the 12-RSS and 23-RSS is CACAGTG, and the native sequence of the nonamers of the 12-RSS and 23-RSS is ACAAAAACC. In non-limiting embodiments, the mutation of the plasmid construct does not include the CAC sequence of the heptamer. In non-limiting embodiments, the plasmid construct mutation may comprise two to ten base pair changes spanning at least a portion of the heptamer and/or at least a portion of the spacer of the 12-RSS or 23-RSS. In non-limiting embodiments, the mutation of the plasmid construct may comprise two to ten base pair changes spanning at least a portion of the spacer and/or at least a portion of the nonamer of the 12-RSS or 23-RSS. In non-limiting embodiments, the mutation of the plasmid construct may comprise the AGTG positions of the heptamer and at least a portion of the spacer of the 12-RSS or 23-RSS. In non-limiting embodiments, the mutation of the plasmid construct may comprise one to ten consecutive base pair changes in the 12-RSS when compared to the native 12-RSS sequence, and one to ten consecutive base pair changes in the 23-RSS when compared to the native 23-RSS sequence.

Certain non-limiting embodiments of the present disclosure are directed to kits that include any of the compositions or library disclosed or otherwise contemplated herein. The kits of the present disclosure may be provided with additional reagents that are used in any of the reactions and/or detection assays of the methods. For example, but not by way of limitation, the kits may include one or more primers, one or more polymerases, one or more restriction enzymes, one or more expression vectors encoding RAG(s), one or more positive or negative controls, and the like, as well as any combinations thereof.

### EXAMPLES

Examples are provided hereinbelow. However, the present disclosure is to be understood to not be limited in its application to the specific experimentation, results, and laboratory procedures disclosed herein. Rather, the Examples are simply provided as one of various embodiments and is meant to be exemplary, not exhaustive.

### Example 1

In this example, V(D)J recombination assembles functional antigen receptor genes from component gene segments to produce the diverse repertoire of functional immunoglobulin and T cell receptors in B and T lymphocytes, respectively. RAG1 and RAG2 are lymphoid-specific proteins that catalyze the DNA cleavage steps in V(D)J recombination. RAG-mediated DNA cleavage activity is directed to discrete DNA sequences known as recombination signal sequences (RSSs) that flank the coding gene segments in the antigen receptor loci. In individual recombination reactions, a heterotetrameric RAG1/2 complex binds simultaneously to two RSSs and creates DNA double strand breaks at the border between each RSS and the adjoining coding segment. Joining of the coding segments is carried out by ubiquitous DNA repair factors. Many RSSs are only semi-conserved, such that recombination of poorly conserved RSSs requires promiscuous RAG1/2 activity. RAG1/2 also creates aberrant recombination events at RSS-like sites, called cryptic RSSs (cRSS), located outside of the antigen receptor loci, which can cause oncogenic chromosomal rearrangements. Therefore, RAG1/2 must be promiscuous to facilitate recombination of poorly conserved RSSs, but it must also be precise to avoid off-target cRSSs. To characterize the DNA sequence specificity of RAG1/2, a high-throughput plasmid recombination method has been developed to analyze V(D)J recombination sequence specificity. Greater than 4000 extrachromosomal V(D)J recombination substrates of differing sequences were transfected into RAG1/2 expressing cells, and the resulting recombination products were selectively amplified and subsequently analyzed by next-generation sequencing. Using this method, RSS motifs that enhance RAG1/2 activity are empirically characterized to shape a diverse antigen receptor repertoire, as well as identify suboptimal RSS motifs that favor nonconventional V(D)J recombination reactions. To date, highly informative results have been obtained from preliminary studies using this method, which indicate that sequence interdependencies exist between different regions of the RSS with significant consequences on the level of V(D)J recombination activity. Furthermore, specific RSS motifs appear to preferentially favor nonconventional V(D)J recombination reactions. The results indicate that specific interrelationships within RSSs: 1) influence their relative utilization by the RAG proteins and 2) govern their fate in conventional versus aberrant V(D)J recombination reactions. The compositions and methods of the present disclosure allow for the analysis of separate regions within the RSS for their effect on V(D)J recombination activity, and the identification of RSS motifs that skew the V(D)J recombination reaction to the formation of aberrant products. Overall, the findings from the methods of present disclosure will significantly improve our current understanding of RAG selectivity of RSSs and cRSSs in normal and aberrant V(D)J recombination reactions, respectively.

In developing B and T lymphocytes, functional antigen receptor genes are assembled from component gene segments by V(D)J recombination through a DNA cleavage and joining mechanism. In this Example, a cellular recombination assay is coupled with a high throughput sequencing approach to decipher patterns in DNA sequences that govern the efficacy of V(D)J recombination. Findings from the methods of the present disclosure are important for elucidating how the antigen receptor repertoire in the adaptive immune system is formed, as well as the basis for aberrant recombination reactions that can lead to oncogenic chromosomal rearrangements.

### Technical Description:

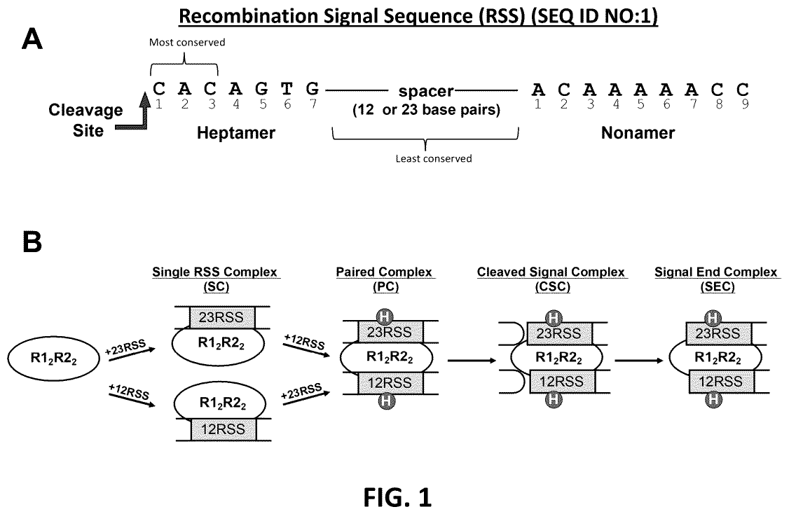

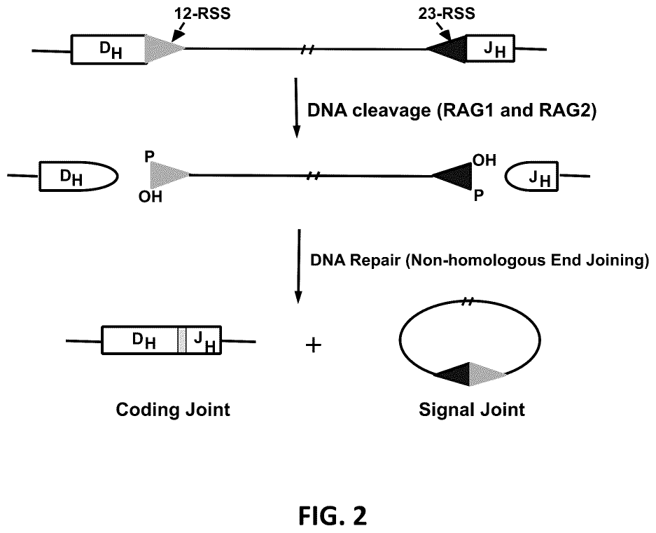

1. Background: In antigen receptor loci, each V, D, and J gene segment is flanked by either one or two RSSs. There are two types of RSSs, known as the 12-RSS and 23-RSS. Each RSS contains a so-called heptamer and nonamer sequence separated by 12 or 23 base pairs. The accepted canonical sequences for the heptamer and nonamer are shown in Panel A of FIG. 1. V(D)J recombination occurs between two gene segments that are flanked by RSSs of differing type. The RAG1 and RAG2 proteins form a heterotetrameric complex that simultaneously binds a 12-RSS and a 23-RSS (FIG. 1, Panel B), and subsequently forms DNA double strand breaks at the borders of each RSS and its flanking gene (coding) segment to form two coding ends and two signal ends, as shown schematically in FIG. 2. The DNA repair factors in non-homologous end joining subsequently join together the two coding ends and the two signal ends to form a coding joint and a signal joint, respectively. The signal joint is typically a precise junction of the RSSs head-to-head, and is a molecular signature of V(D)J recombination.

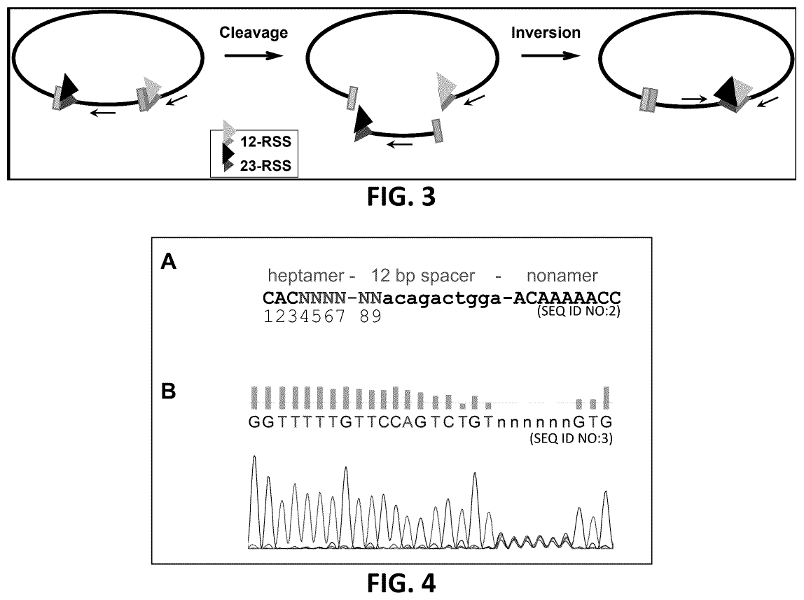

2. Method: The SARP-seq method includes an extrachromosomal recombination assay for V(D)J recombination activity, where the plasmid substrate contains a 12-RSS and a 23-RSS sequence in a co-directional orientation. The plasmid substrate was co-transfected into non-lymphoid cells with RAG1 and RAG2 expression vectors. Subsequently, the cells were cultured for 2-4 days, then the cells were harvested and lysed and the plasmid recovered. Recombined plasmid resulted in inversion of a section of the plasmid to yield a signal joint (FIG. 3).

3. In the SARP-seq method, the plasmid pMX-INV (containing a canonical 12-RSS and 23-RSS, and also referred to as pMX-RSS-EGFP/IRES-hCD4) was modified by introducing a window of 6 consecutive fully degenerate base pairs into the 12-RSS through directional ligation to form the pHR library. The pHR library was generated entirely in vitro. Bacterial transformation was avoided to preserve the degeneracy of the 6 bp region of interest in the resulting plasmid library. Steps 4-9 outline production of the pHR library.

4. The pMX-INV plasmid was digested with MluI and EcoRI restriction endonucleases to linearize the plasmid and remove the existing canonical 12-RSS. The linearized plasmid was gel purified.

5. A synthetic single-stranded oligonucleotide (ordered and obtained from Integrated DNA Technologies, Coralville, IA) contained the 12-RSS and flanking sequences. The 12-RSS contained 6 degenerate bases (FIG. 4, Panel A). The flanking sequences contained sequences immediately 5′ to the 12-RSS necessary for next generation sequencing using ILLUMINA® sequencing platforms (ILLUMINA®, Inc., San Diego, CA). Two restriction endonuclease sites, an MluI site and an EcoRI site, were incorporated near the 5′ and 3′ ends of the oligonucleotide, respectively.

6. The synthetic oligonucleotide in #5 was converted to double-stranded DNA through a primer extension reaction. The resulting double-stranded DNA was incubated with MluI and EcoRI, and subsequently gel purified.

7. The DNA fragment from #6 was ligated to the linearized pMX-INV plasmid in #4.

8. The ligation product was gel purified. An aliquot of the purified pHR library was sequenced by capillary DNA sequencing to confirm that the 12-RSS contained the 6 consecutive degenerate base pairs (FIG. 4, Panel B). The resulting product was the input library for the extrachromosomal recombination assay. The pHR library (the input library) theoretically contains 4096 sequences (4⁶). However, the two sequences containing either the MluI or EcoRI sites will be poorly represented. The remaining 4094 sequences are expected to be represented at approximately equivalent levels.

9. Alternative methods are available and can be utilized to generate the plasmid with the degenerate DNA; non-limiting examples thereof include using Gibson Assembly method, alternate restriction enzyme sites, or PCR methods. Regardless of the method utilized, it is recommended that the quality of the plasmid input library be confirmed by capillary DNA sequencing.

10. The input plasmid library was transfected into HEK293T cells along with expression vectors for RAG1 and RAG2. The expression vectors encoded for Cherry fluorescent protein fused to the core region of RAG2 and maltose binding protein (MBP) fused to the core region of RAG1. Alternatively, pre-lymphocytes can be used; in this instance, endogenous RAG1/2 expression can be induced, thereby eliminating the need for transfection with RAG-expressing vectors.

11. Following transfection in step #10, HEK293T cells were cultured in Dulbecco's Modified Eagle Medium (DMEM™) at 37° C. in 5% CO₂ for 72 hours. However, these values are for purposes of example only. The time period that cells are cultured post-transfection can be adjusted and optimized for each cell type and the specific goal of the experiment.

12. After step #11, the cells were harvested and the plasmid DNA recovered using a modified Hirt procedure. The total amount of plasmid DNA recovered was quantified by spectrophotometry or gel electrophoresis.

13. Successful recombination consists of an inversion of the portion of the plasmid between the 12-RSS and 23-RSS (and includes the 23-RSS), resulting in a 12-RSS:23-RSS signal joint. PCR primers designed to prime 5′ to the 12-RSS and within the inverted region will only yield the expected PCR product on recombined plasmids; hence, selective amplification of recombined products (SARP). The relative position of the PCR primers to the recombined product is shown schematically in FIG. 3. If necessary, nested PCR can be performed to increase the purity of the PCR product. The final PCR product contained ILLUMINA® adaptor sequences at both the 5′ and 3′ ends. The template for the sequencing primer was included at 10 base pairs 5′ to the 12-RSS.

14. The PCR product from #13 was gel purified and constituted the output library from the recombination assay.

15. The PCR output library from #14 was subjected to ILLUMINA® sequencing according to the manufacturer's directions. Following sequencing, the quality of the sequencing (Q scores) was analyzed. Q scores >30 were evident for the region of interest, which includes the window of consecutively degenerate base pairs.

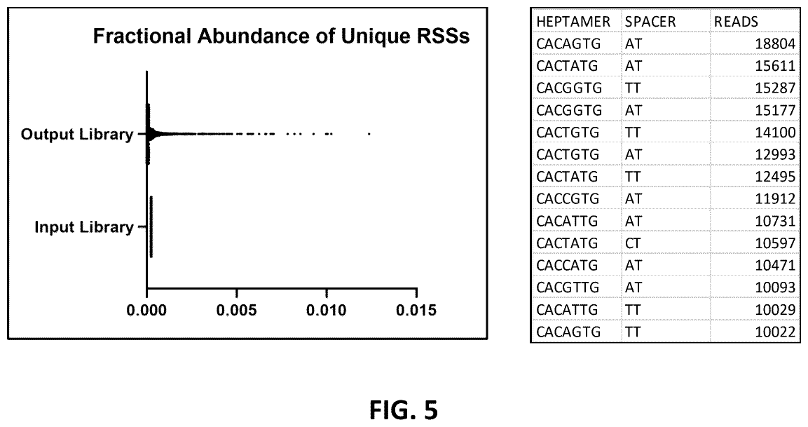

16. Sequences were sorted for the presence of the 12-RSS:23-RSS signal joints, and the number of reads for specific 12-RSS/spacer sequences were tabulated. The top 12% of reads in a tabulated form are shown in FIG. 5.

17. A hierarchy of sequences that are preferentially recombined can be determined (FIG. 5), including identifying preferred sequence motifs. An advantage of the methods of the present disclosure is that thousands, or potentially millions, of sequences can be analyzed simultaneously for recombination activity. As the window of degenerate base pairs is embedded in a constant sequence background, only the effect of DNA sequence within the specified window is interrogated. There is therefore no effect on activity due to variability in flanking sequences.

### Example 2

Example 1 describes the use of a SARP-Seq plasmid construct that contains 6 consecutive degenerate base pairs located at positions 4 to 7 of the 12-RSS heptamer and the 2 adjacent base pairs in the spacer region. Example 1 demonstrated the feasibility of obtaining sequence-specific information using the methods of the present disclosure.

In this Example, the PCR primers utilized in the SARP-Seq have been modified to include barcodes. The experiments are analyzed on the same ILLUMINA® flowcell, and the resulting DNA sequences are indexed by barcode. The PCR primers also include a Unique Molecular Identifier (UMI), a 12 base pair degenerate sequence that is used to identify and eliminate from analysis any PCR overamplification errors.

In another experiment, the fully degenerate window is increased in length from 6 to 8 bp, and is placed at different portions of the 12-RSS or 23-RSS, including the heptamer/spacer and nonamer/spacer regions. Other substrates include nonconsecutive degenerate base pairs located in both the heptamer and nonamer regions. Results from these experiments determine sequence specificity at separate and defined locations of the entire RSS. Deep sequencing also indicates the range of RSSs that are capable of being cleaved by the RAG proteins, and are used in the analysis of genomic abnormalities in suspected RAG-mediated neoplasms.

In another experiment, RAG1 and RAG2 mutants that lead to immune system disorders are used in place of the wild type proteins, to test the range of sequence specificity of the mutants as compared to the wild type proteins.

### Example 3

This Example includes additional uses of the SARP technology described herein. In particular, extensions of the SARP-seq method with regard to protocol modifications, output analysis, and substrate design are given below.

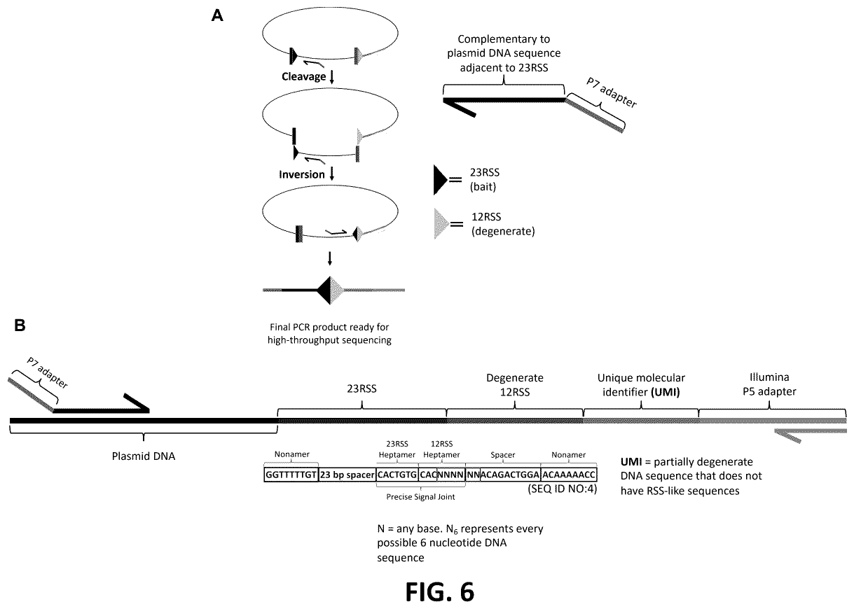

SARP-seq with Unique Molecular Identifier: The incorporation of a Unique Molecular Identifier (UMI) into the output library eliminates misinterpretation of PCR overamplification artifacts that may occur during library preparation. In this modification, the UMI is a partially degenerate DNA sequence incorporated in the input plasmid substrate adjacent to the 12-RSS (FIG. 6). The UMI is designed to contain no potential RSS heptamer-like CAC-containing sequences in order to prevent competition with the target 12-RSS.

The two modifications of the SARP-seq method described herein after will test if certain RSS sequences increase the risk for aberrant V(D)J recombination products, which include imprecise signal joints and hybrid joints.

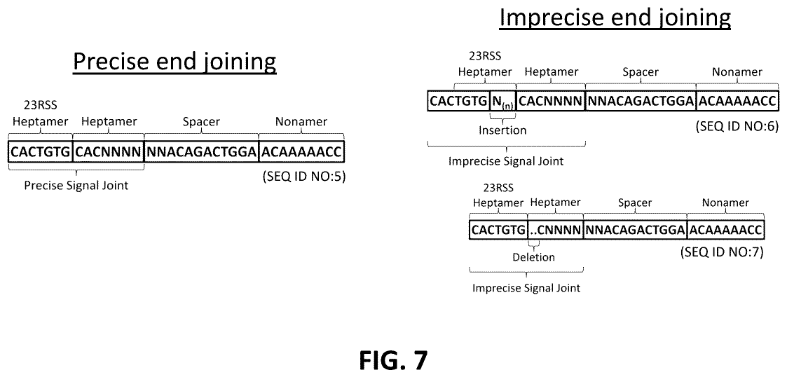

Imprecise Signal Joints: In Step 16 of the method of Example 1, analysis of sequences of the output library (the final PCR product) are sorted and analyzed for precise signal joints, where the 12-RSS and 23-RSS heptamers are joined head-to-head with no addition or deletion of base pairs. Minor amounts of Imprecise signal joints are also formed where bases are deleted or added prior to joining of the signal ends during V(D)J recombination (FIG. 7). Preliminary results indicate that certain 12-RSSs are found in a higher number of reads that contain imprecise versus precise signal joint products.

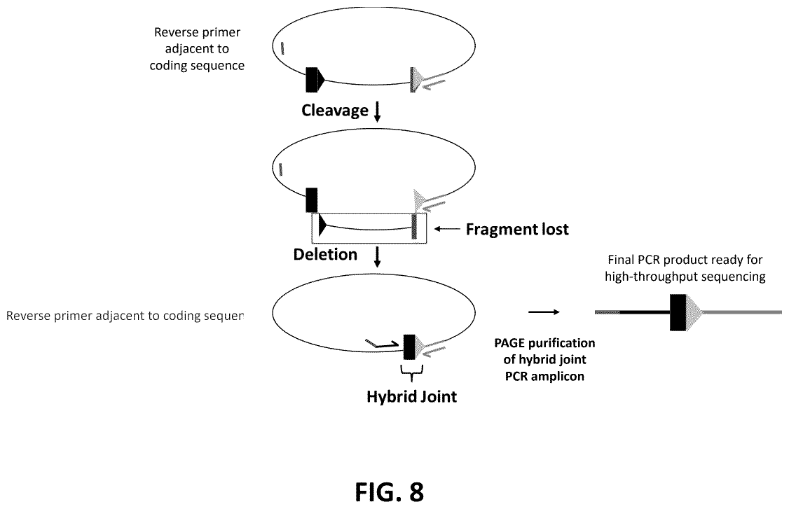

Hybrid Joints: Another aberrant V(D)J recombination product is the hybrid joint. In contrast to the signal joint, the hybrid joint is where each RSS is joined to the DNA that previously bordered the partner RSS. In the plasmid substrate used in the SARP-seq method, this results in deletion, rather than inversion, of a fragment from the input substrate (FIG. 8). Sequence analysis of the hybrid joint-containing output library will show if certain RSS sequences are preferentially found in hybrid joint formation.

Design variations of the SARP-seq input plasmid substrate: The SARP-seq method is flexible in its design where different regions of the RSS can be examined. Examples include increasing the number of consecutive degenerate bases (i.e., up to 9 bases instead of the 6 bases in FIG. 4, Panel A). In addition, the degenerate bases can be incorporated at different positions in the RSS, such as in the nonamer.

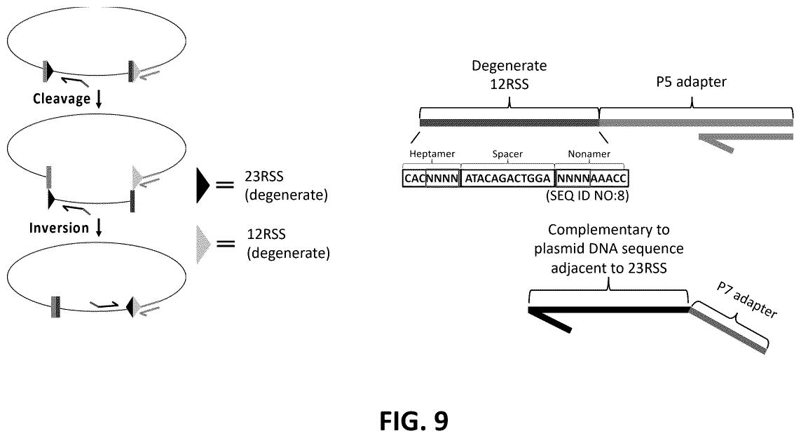

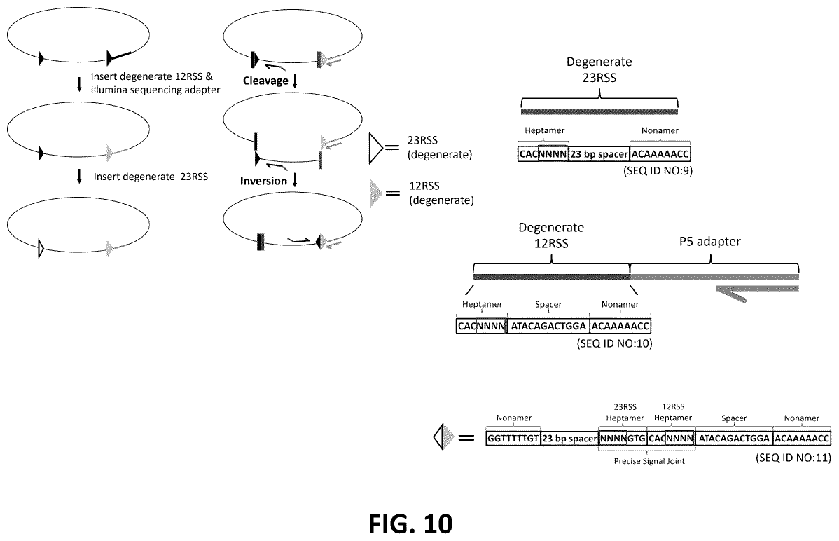

More complex designs include where both the heptamer and nonamer regions contain degenerate bases (FIG. 9). This example tests if interrelationships exist between these regions of the RSS, which affect the efficacy of the V(D)J recombination reaction. The degenerate regions may also be split between the 12-RSS and the 23-RSS to test if there were paired preferences between the partner RSSs (FIG. 10). The plasmid containing degenerate DNA in both RSSs is constructed in a two-step process. First, the 12-RSS-containing degenerate DNA is inserted into the plasmid as in the original SARP-seq protocol to form library 1 (see steps 4-9 of Example 1). Second, the 23-RSS-containing degenerate DNA is inserted into library 1 to create library 2, yielding the final input library for the V(D)J recombination reaction. The production of the output library is as described in the Technical Description of the original protocol (see steps 13-15 of Example 1).

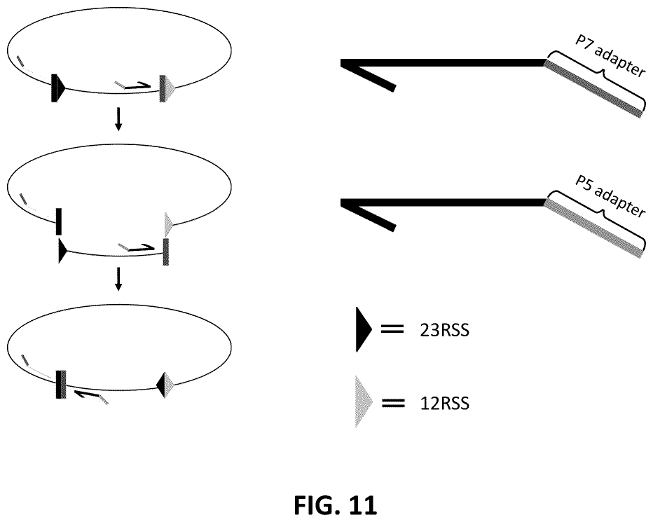

Analysis of Coding Joint Formation: Besides analysis of the signal joint, the coding joint can be analyzed to determine the extent of sequence variation in the coding joint produced by NHEJ (an inaccurate DNA repair pathway). Coding joint variability is a hallmark of V(D)J recombination, but has typically been studied in low throughput sequencing methods. In FIG. 11, 12-RSS and 23-RSS of defined sequences that do not contain degenerate bases are used, and PCR primers are designed to amplify the coding joint of the plasmid of the recombined plasmids. The PCR product is subsequently analyzed by next generation sequencing (NGS). This variation of SARP-seq can be used to test the role of individual factors in the nonhomologous end joining (NHEJ) DNA repair step of V(D)J recombination.

While the attached disclosures describe the inventive concept(s) in conjunction with the specific drawings, experimentation, results, and language set forth hereinafter, it is evident that many alternatives, modifications, and variations will be apparent to those skilled in the art. Accordingly, it is intended to embrace all such alternatives, modifications, and variations that fall within the spirit and broad scope of the present disclosure.

What is claimed is:

1. A high throughput method of analyzing DNA sequence-specificity in a V(D)J recombination assay, the method comprising the steps of:
producing a plasmid library comprising a plurality of plasmid constructs, each plasmid construct comprising a plasmid vector having a 12-recombination signal sequence (12-RSS) and a 23-recombination signal sequence (23-RSS) inserted therein in a colinear orientation and a segment of at least 100 base pairs inserted in between the 12-RSS and the 23-RSS, wherein the 12-RSS comprises a heptamer beginning with a sequence of CAC, a 12 base pair spacer, and a nonamer, and wherein the 23-RSS comprises a heptamer beginning with a sequence of CAC, a 23 base pair spacer, and a nonamer, wherein at least one of the 12-RSS and the 23-RSS in each plasmid construct has a degenerate base pair sequence of one to ten consecutive base pairs present therein that does not include the CAC sequence of the heptamers, and wherein the plasmid library comprises about 4ⁿ plasmids, wherein n is the number of base pairs present in the degenerate base pair sequence, and wherein the method of producing the plasmid library comprises the steps of:
producing a first plurality of synthetic oligonucleotides that comprise the 12-RSS having the degenerate base pair sequence of one to ten consecutive base pairs present therein, wherein the first plurality of synthetic oligonucleotides comprises about 4ⁿ oligonucleotides, wherein n is the number of base pairs present in the degenerate base pair sequence;
producing a second plurality of synthetic oligonucleotides that comprise the 23-RSS having the degenerate base pair sequence of one to ten consecutive base pairs present therein, wherein the second plurality of synthetic oligonucleotides comprises about 4ⁿ oligonucleotides, wherein n is the number of base pairs present in the degenerate base pair sequence;
converting the first and second pluralities of synthetic oligonucleotides to double-stranded DNA;
linearizing a plasmid, wherein the plasmid comprises canonical 12-RSS and canonical 23-RSS present therein in a colinear orientation and the segment of at least 100 base pairs disposed therebetween;
removing the canonical 12-RSS and 23-RSS; and
ligating the double-stranded DNA comprising the first and second pluralities of synthetic oligonucleotides to the plasmid to produce the plasmid library;
transfecting mammalian cells with the plasmid library, wherein the mammalian cells are capable of expressing recombination activating gene proteins 1 and 2 (RAG1 and RAG2);
culturing the transfected cells under conditions that allow for expression of RAG1 and RAG2 and production of recombination products in which the portion of the plasmids between the 12-RSS and 23-RSS is inverted to form a 12-RSS:23-RSS signal joint and a coding joint;
harvesting the transfected mammalian cells;
recovering plasmid DNA from the harvested cells; and
selectively amplifying the recombination products using primers that amplify the 12-RSS:23-RSS signal joint formed during recombination, wherein the selectively amplified recombination products constitute an output library for the recombination assay.

2. The method of claim 1, further comprising the steps of:
sequencing the output library from the recombination assay; and
analyzing the degenerate base pair sequences present in the selectively amplified signal joints of the output library.

3. The method of claim 1, wherein the mammalian cells endogenously express RAG1 and RAG2 and/or have been transfected with at least one expression vector encoding RAG1 and at least one expression vector encoding RAG2, and/or at least one expression vector encoding both RAG1 and RAG2.

4. The method of claim 1, wherein at least one of the RAG1 and RAG2 comprises at least one mutation therein, and wherein the method is further defined as a method of analyzing DNA sequence-specificity of the RAG mutant in a V(D)J recombination assay.

5. The method of claim 1, wherein the plasmid vector utilized in the plasmid library is pMX-INV.

6. The method of claim 1, wherein the degenerate base pair sequence comprises two to ten consecutive base pairs that span at least a portion of the heptamer and at least a portion of the spacer of the 12-RSS and/or 23-RSS.

7. The method of claim 1, wherein the degenerate base pair sequence comprises two to ten base pair changes that span at least a portion of the spacer and at least a portion of the nonamer of the 12-RSS and/or 23-RSS.

8. The method of claim 1, wherein the degenerate base pair sequence comprises the positions of the canonical AGTG sequence of the heptamer and at least a portion of the spacer of the 12-RSS and/or 23-RSS.

9. The method of claim 1, wherein the degenerate base pair sequence has one consecutive base pair present therein.

10. The method of claim 1, wherein the degenerate base pair sequence has two consecutive base pairs present therein.

11. The method of claim 1, wherein the degenerate base pair sequence has three consecutive base pairs present therein.

12. The method of claim 1, wherein the degenerate base pair sequence has four consecutive base pairs present therein.

13. The method of claim 1, wherein the degenerate base pair sequence has five consecutive base pairs present therein.

14. The method of claim 1, wherein the degenerate base pair sequence has six consecutive base pairs present therein.

15. The method of claim 1, wherein the degenerate base pair sequence has seven consecutive base pairs present therein.

16. The method of claim 1, wherein the degenerate base pair sequence has eight consecutive base pairs present therein.

17. The method of claim 1, wherein the degenerate base pair sequence has nine consecutive base pairs present therein.

18. The method of claim 1, wherein the degenerate base pair sequence has ten consecutive base pairs present therein.

19. A high throughput method of analyzing DNA sequence-specificity in a V(D)J recombination assay, the method comprising the steps of:
producing a plasmid library comprising a plurality of plasmid constructs, each plasmid construct comprising a plasmid vector having a 12-recombination signal sequence (12-RSS) and a 23-recombination signal sequence (23-RSS) inserted therein in a colinear orientation and a segment of at least 100 base pairs inserted in between the 12-RSS and the 23-RSS, wherein the 12-RSS comprises a heptamer beginning with a sequence of CAC, a 12 base pair spacer, and a nonamer, and wherein the 23-RSS comprises a heptamer beginning with a sequence of CAC, a 23 base pair spacer, and a nonamer, wherein at least one of the 12-RSS and the 23-RSS in each plasmid construct has a degenerate base pair sequence of one to six consecutive base pairs present therein that does not include the CAC sequence of the heptamers, and wherein the plasmid library comprises about 4ⁿ plasmids, wherein n is the number of base pairs present in the degenerate base pair sequence, and wherein the method of producing the plasmid library comprises the steps of:
producing a first plurality of synthetic oligonucleotides that comprise the 12-RSS having the degenerate base pair sequence of one to six consecutive base pairs present therein, wherein the first plurality of synthetic oligonucleotides comprises about 4ⁿ oligonucleotides, wherein n is the number of base pairs present in the degenerate base pair sequence;
producing a second plurality of synthetic oligonucleotides that comprise the 23-RSS having the degenerate base pair sequence of one to six consecutive base pairs present therein, wherein the second plurality of synthetic oligonucleotides comprises about 4ⁿ oligonucleotides, wherein n is the number of base pairs present in the degenerate base pair sequence;
converting the first and second pluralities of synthetic oligonucleotides to double-stranded DNA;
linearizing a plasmid, wherein the plasmid comprises canonical 12-RSS and canonical 23-RSS present therein in a colinear orientation and the segment of at least 100 base pairs disposed therebetween;
removing the canonical 12-RSS and 23-RSS; and
ligating the double-stranded DNA comprising the first and second pluralities of synthetic oligonucleotides to the plasmid to produce the plasmid library;
transfecting mammalian cells with the plasmid library, wherein the mammalian cells are capable of expressing recombination activating gene proteins 1 and 2 (RAG1 and RAG2);
culturing the transfected cells under conditions that allow for expression of RAG1 and RAG2 and production of recombination products in which the portion of the plasmids between the 12-RSS and 23-RSS is inverted to form a 12-RSS:23-RSS signal joint and a coding joint;
harvesting the transfected mammalian cells;
recovering plasmid DNA from the harvested cells; and
selectively amplifying the recombination products using primers that amplify the 12-RSS:23-RSS signal joint formed during recombination, wherein the selectively amplified recombination products constitute an output library for the recombination assay.

20. The method of claim 19, further comprising the steps of:
sequencing the output library from the recombination assay; and
analyzing the degenerate base pair sequences present in the selectively amplified signal joints of the output library.


SEQUENCE LISTING (11 sequences)

SEQ ID NO: 1 - DNA, 39 bp, Homo sapiens
  misc_feature 8..30: note = Each n can be any nucleotide. Also, residues 8-30 represents a spacer that can be either 12 or 23 base pairs long. As such, SEQ ID NO:1 includes the 39 residue sequence as well as a 28 residue sequence in whch residues 20 to 30 are removed.
  source 1..39: mol_type = other DNA
cacagtgnnnnnnnnnnnnnnnnnnnnnnnacaaaaacc

SEQ ID NO: 2 - DNA, 28 bp, synthetic construct
  misc_feature 1..28: note = synthetic single-stranded oligonucleotide containing the 12-RSS and flanking sequences, wherein the 12-RSS contained 6 degenerate bases
  misc_difference 4..9: note = n is a, c, g, or t
  source 1..28: mol_type = other DNA
cacnnnnnnacagactggaacaaaaacc

SEQ ID NO: 3 - DNA, 28 bp, synthetic construct
  misc_feature 1..28: note = Capillary DNA sequencing data of the plasmid input library based on the degenerate sequences of SEQ ID NO:4
  misc_difference 20..25: note = n is a, c, g, or t
  source 1..28: mol_type = other DNA
ggtttttgttccagtctgtnnnnnngtg

SEQ ID NO: 4 - DNA, 67 bp, synthetic construct
  misc_feature 1..67: note = final PCR product of output library following V(D)J recombination
  misc_difference 10..32: note = n is a, c, g, or t
  misc_difference 43..48: note = n is a, c, g, or t
  source 1..67: mol_type = other DNA
ggtttttgtnnnnnnnnnnnnnnnnnnnnnnncactgtgcacnnnnnnacagactggaacaaaaacc

SEQ ID NO: 5 - DNA, 35 bp, synthetic construct
  misc_feature 1..35: note = precise end joining sequence of variant RSS
  misc_difference 11..16: note = n is a, c, g, or t
  source 1..35: mol_type = other DNA
cactgtgcacnnnnnnacagactggaacaaaaacc

SEQ ID NO: 6 - DNA, 36 bp, synthetic construct
  misc_feature 1..36: note = Imprecise end joining sequence of variant RSS
  misc_difference 8: note = n is a, c, g, or t
  misc_difference 12..17: note = n is a, c, g, or t
  source 1..36: mol_type = other DNA
catcgtgncacnnnnnnacagactggaacaaaaacc

SEQ ID NO: 7 - DNA, 33 bp, synthetic construct
  misc_feature 1..33: note = precise end joining sequence of variant RSS
  misc_difference 9..14: note = n is a, c, g, or t
  source 1..33: mol_type = other DNA
catcgtgcnnnnnnacagactggaacaaaaacc

SEQ ID NO: 8 - DNA, 27 bp, synthetic construct
  misc_feature 1..27: note = Synthetic oligonucleotide of degenerate 12RSS where both the heptamer and nonamer regions contain degenerate bases
  misc_difference 4..7: note = n is a, c, g, or t
  misc_difference 20..23: note = n is a, c, g, or t
  source 1..27: mol_type = other DNA
cacnnnnatacagactggannnnaacc

SEQ ID NO: 9 - DNA, 17 bp, synthetic construct
  misc_feature 1..17: note = Synthetic oligonucleotide containing degenerate 23RSS
  misc_difference 4..8: note = n is a, c, g, or t
  source 1..17: mol_type = other DNA
cacnnnnnacaaaaacc

SEQ ID NO: 10 - DNA, 28 bp, synthetic construct
  misc_feature 1..28: note = Synthetic oligonucleotide containing degenerate 12RSS
  misc_difference 4..7: note = n is a, c, g, or t
  source 1..28: mol_type = other DNA
cacnnnnatacagactggaacaaaaacc

SEQ ID NO: 11 - DNA, 45 bp, synthetic construct
  misc_feature 1..45: note = sequence of plasmid containing degenerate DNA in both RSSs, as in SEQ ID NOS:25-26
  misc_difference 10..14: note = n is a, c, g, or t
  misc_difference 21..24: note = n is a, c, g, or t
  source 1..45: mol_type = other DNA
ggtttttgtnnnnngtgcacnnnnatacagactggaacaaaaacc